(19) United States
(12) Patent Application Publication
(10) Pub. No.: US 2026/0225977 A1
(43) Pub. Date: Aug. 6, 2026

(54) CO-PRODUCTION OF 1,1,1-TRIFLUOROPROPENE (1243ZF) AND E-1,1,1,4,4,4-HEXAFLUORO-2-BUTENE (E-1336MZZ)

(71) Applicant: THE CHEMOURS COMPANY FC, LLC, WILMINGTON, DE (US)

(72) Inventors: SHENG PENG, HOCKESSIN, DE (US); MARIO JOSEPH NAPPA, LEEBURG, FL (US); MICHAEL A. BRADLEY, HOCKESSIN, DE (US)

(73) Assignee: THE CHEMOURS COMPANY FC, LLC, WILMINGTON, DE (US)

(21) Appl. No.: 19/148,463

(22) PCT Filed: Jan. 30, 2024
(86) PCT No.: PCT/US2024/013421

§ 371 Date: Jul. 16, 2025

Related U.S. Application Data
(60) Provisional application No. 63/441,970, filed on Jan. 30, 2023.

(51) Int. Cl. C07C 17/20; C07C 21/18

(52) U.S. Cl. CPC C07C 17/206; C07C 21/18

(57) ABSTRACT

The present invention relates to processes for co-producing 1,1,1-trifluoropropene (HFO-1243zf) and E-1,1,1,4,4,4-Hexafluoro-2-butene (E-1336mzz).

### CROSS REFERENCE TO RELATED APPLICATIONS

This application is national filing under 35 U.S.C. 371 of International Application Number PCT/US2024/013421 filed Jan. 30, 2024, and claims the benefit of priority of U.S. Provisional Application No. 63/441,970 filed Jan. 30, 2023, the disclosures of which are incorporated herein by reference in its entirety.

### BACKGROUND OF THE INVENTION

Many industries have been working for the past few decades to find replacements for the ozone depleting chlorofluorocarbons (CFCs) and hydrochlorofluorocarbons (HCFCs). The CFCs and HCFCs have been employed in a wide range of applications, including their use as aerosol propellants, refrigerants, cleaning agents, expansion agents for thermoplastic and thermoset foams, heat transfer media, immersion fluid for electronics, gaseous dielectrics, fire extinguishing and suppression agents, power cycle working fluids, polymerization media, particulate removal fluids, carrier fluids, buffing abrasive agents, and displacement drying agents. In the search for replacements for these versatile compounds, many industries have turned to the use of hydrofluorocarbons (HFCs), and more recently hydrochlorofluoroolefins (HCFOs) and hydrofluoroolefins (HFOs) which have lower global warming potentials (GWP), and C₃-C₆ hydro(chloro)fluoroolefins with higher boiling point making them especially suitable for the electronics industry.

Many of the hydrochlorofluoroolefins (HCFOs) and hydrofluoroolefins (HFOs) which have lower global warming potentials (GWP) are prepared from precursor material including but not limited to 1,1,1-trifluorpropene (TPY), HFO-1243zf or, 1243zf). HFO-1243zf is also useful as a monomer for telomerization, copolymerization, and production of other compounds, including, but not limited to fluorosilicones and pharmaceutical. Thus whether a monomer or an intermediate compound for the production of hydrochlorofluorocarbons (HCFCs), hydrofluorocarbons (HFCs), hydrochlorofluoroolefins (HCFOs) and hydrofluoroolefins (HFOs), a need remains for more effective and efficient ways of producing and using hydrochlorofluoroolefins (HCFOs) and hydrofluoroolefins (HFOs).

Processes for producing C₃-C₆ hydro(chloro)fluoroolefins generally involve using precursor or intermediate compounds, such as trifluoropropene (HFO-1243zf) which is commercially available or can be produced involving a two-step process according to the following reaction scheme:

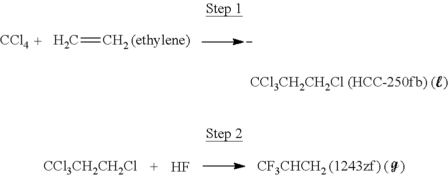

Generally, steps 1 and 2 are carried out in separate reactors and require HCC-250fb to be separated prior to fluorination.

The 1243zf intermediate is also used to produce (E/Z)-1,1,1,4,4,4-hexafluorobut-2-ene((E/Z)-HFO-1336mzz) which is suitable for use as a working fluid in heat transfer systems including, but are not limited to, air conditioners (e.g., automotive air conditioners), freezers, refrigeration machines, heat pumps, chillers (e.g., water chillers, flooded evaporator chillers, direct expansion chillers, centrifugal chillers), walk-in chillers, high temperature heat pumps, mobile chillers, mobile air conditioning units, immersion cooling systems, data center cooling systems, and combinations thereof, and as a component of compositions for heat transfer, refrigeration, high-temperature heat pumps, immersion cooling systems, organic Rankine cycles, fire extinguishing/fire suppression agents, propellants, foam blowing agents, solvents, and/or cleaning fluids.

One reaction scheme for producing HFO-1336mzz involves four process steps and separate reactors.

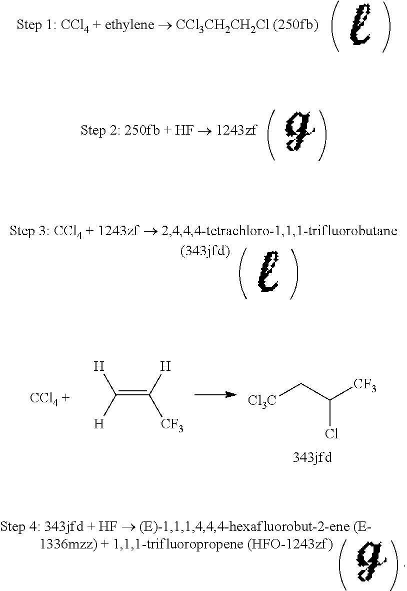

It would be advantageous if the process for producing (E)-1,1,1,4,4,4-hexafluorobut-2-ene (E-1336mzz) could be simplified to reduce investment cost and increase process efficiency (time and yield) without the capital expenditures and loss of time incurred with the four-step process.

### SUMMARY OF THE INVENTION

The present invention provides a simplified process for co-producing 1,1,1-trifluoropropene (HFO-1243zf) and E-1,1,1,4,4,4-hexafluoro-2-butene (E-1336mzz).

The present invention provides a process for contacting 250fb and 343jfd feed streams in a catalytic reaction zone with HF to co-produce 1,1,1-trifluoropropene (HFO-1243zf) and E-1,1,1,4,4,4-hexafluoro-2-butene (E-1336mzz).

The present invention provides an integrated process using co-feed which is fluorinated in a single vessel to co-produce 1243zf and E-HFO-1336mzz, according to the following reaction schemes:

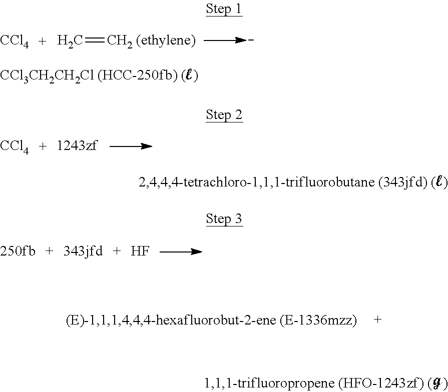

The present invention provides a process for concurrently producing 250fb and 343jfd, forming 250fb and 343jfd feed streams, and contacting the feed streams and hydrogen fluoride, in the presence of a catalyst, to produce E-1,1,1,4,4,4-hexafluoro-2-butene (E-1336mzz) from a co-feed in a single reactor.

The present invention provides a process of contacting HCC and HCFO compounds in the presence of a catalyst, in the gas phase, and forming 1,1,1-trifluoropropene (HFO-1243zf) and E-1,1,1,4,4,4-hexafluoro-2-butene (E-1336mzz), wherein the HCFO comprises 2,4,4,4-tetrachloro-1,1,1-trifluorobutane (343jfd) and the HCC comprises CCl₃CH₂CH₂Cl (HCC-250fb).

The present invention relates to compositions comprising one of (1) 1,1,1-trifluoropropene (HFO-1243zf) and E-1,1,1,4,4,4-hexafluoro-2-butene (E-HFO-1336mzz) or (2) 2,4,4,4-tetrachloro-1,1,1-trifluorobutane (343jfd), CCl₃CH₂CH₂Cl (HCC-250fb), 1,1,1-trifluoropropene (HFO-1243zf) and E-1,1,1,4,4,4-hexafluoro-2-butene (E-1336mzz), optionally including at least one additional member selected from 346mdf, 253fb, 1242zf, 1336 ft, 356mff, Z-1326mxz, 1233xf, E-1233zd, 252da, 1241 isomer, and 1333azd.

The present invention relates to compositions comprising one of (1) 1,1,1-trifluoropropene (HFO-1243zf) and E-1,1,1,4,4,4-hexafluoro-2-butene (E-HFO-1336mzz), optionally including at least one additional member selected from 346mdf, 253fb, 1242zf, 1336 ft, 356mff, Z-1326mxz, 1233xf, E-1233zd, Z-1336mxx, 252da, 1241 isomer, and 1333azd.

The present invention relates to compositions comprising 2,4,4,4-tetrachloro-1,1,1-trifluorobutane (343jfd), CCl₃CH₂CH₂Cl (HCC-250fb) and HF.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 depicts a flow process for an embodiment of the invention.

### DETAILED DESCRIPTION OF THE INVENTION

While HFCs do not contribute to the destruction of stratospheric ozone, they are of concern due to their contribution to the “greenhouse effect”, i.e., they contribute to global warming. As a result of their contribution to global warming, the HFCs have come under scrutiny, and their widespread use may also be limited in the future. Thus, there is a need for hydrofluoroolefins, such as 1,1,1,4,4,4-hexafluoro-2-butene (CF₃CH═CHCF₃, HFO-1336mzz), which is suitable for use as a working fluid in heat transfer systems including, including but not limited to, air conditioners (e.g., automotive air conditioners), freezers, refrigeration machines, heat pumps, chillers (e.g., water chillers, flooded evaporator chillers, direct expansion chillers, centrifugal chillers), walk-in chillers, high temperature heat pumps, mobile chillers, mobile air conditioning units, immersion cooling systems, data center cooling systems, and combinations thereof, and as a component of compositions for heat transfer, refrigeration, high-temperature heat pumps, immersion cooling systems, organic Rankine cycles, fire extinguishing/fire suppression agents, propellants, foam blowing agents, solvents, and/or cleaning fluids, which possess high efficiency, and thermal stability meets both goals. Similarly, there is a need for simplified processes for producing HFO-1243zf which is a key intermediate for producing HFO-1336mzz, as well as many other HFC, HFO and HCFO products.

### Definitions

Where applicants have defined an invention or a portion thereof with an open-ended term such as “comprising,” it should be readily understood that (unless otherwise stated) the description should be interpreted to also include such an invention using the terms “consisting essentially of” or “consisting of.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method that includes materials, steps, features, components, or elements, in addition to those literally disclosed provided that these additional included materials, steps, features, components, or elements that do not materially affect the basic and novel characteristic(s) of the claimed invention, especially the mode of action to achieve the desired result of any of the processes of the present invention. The term ‘consisting essentially of’ occupies a middle ground between “comprising” and ‘consisting of’.

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

As used herein, the term “about” is meant to account for variations due to experimental error (e.g., plus or minus approximately 10% of the indicated value). All measurements reported herein are understood to be modified by the term “about”, whether or not the term is explicitly used, unless explicitly stated otherwise.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed.

Where a range of numerical values is recited herein, unless otherwise stated, the range is intended to include the endpoints thereof, and all integers and fractions within the range.

Global warming potential (GWP) is an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100-year time horizon is commonly the value referenced.

As used herein, “absence of HF” means that a constant flow of HF is not present during the reaction, but not does exclude use of HF to activate the catalyst prior to the reaction.

As used herein organophosphine and organophosphorus are used synonymously and can include primary, second and tertiary phosphines.

The compounds described herein can be asymmetric (e.g., having one or more stereocenters). All stereoisomers, such as enantiomers and diastereomers, are intended unless otherwise indicated. Cis/trans and/or E/Z geometric isomers of the compounds of the present invention are described and may be isolated as a mixture of isomers or as separated isomeric forms.

### Chemicals, Abbreviations, and Acronyms

| ID | Formula | Chemical Name |
|---|---|---|
| HCFC-343jfd | CCl₃CH₂CHClCF₃ | 2,4,4,4-tetrachloro-1,1,1- |
| | | trifluorobutane |
| R-1336mzz or HFO- | CF₃CH═CHCF₃ | 1,1,1,4,4,4-hexafluorobut-2-ene |
| 1336mzz | | |
| E-1336mzz or (E)- | E-CF₃CH═CHCF₃ | (E)-1,1,1,4,4,4-hexafluorobut-2-ene |
| HFO-1336mzz | | |
| Z-1336mzz or (Z)- | Z-CF₃CH═CHCF₃ | (Z)-1,1,1,4,4,4-hexafluorobut-2-ene |
| HFO-1336mzz | | |
| HCFO-1333azd | CCl₂═CHCHClCF₃ | 1,1,3-trichloro-4,4,4-trifluorobut-1- |
| | | ene |
| HFO-1336ft | CF₃(CF₃)C═CH₂ | 3,3,3-trifluoro-2- |
| | | (trifluoromethyl)prop-1-ene |
| HCFC-346mdf | CF₃CH₂CHClCF₃ | 2-chloro-1,1,1,4,4,4- |
| | | hexafluorobutane |
| HCFO-1335lzz | CF₂ClCH═CHCF₃ | 1-chloro-1,1,4,4,4-pentafluorobut-2- |
| | | ene |
| HFC-356mff | CF₃CH₂CH₂CF₃ | 1,1,1,4,4,4-hexafluorobutane |
| HCFO-1326mxz | CF₃CCl═CHCF₃ | 2-chloro-1,1,1,4,4,4-hexafluoro-2- |
| | | butene |
| HCFO-1241 isomer | C₃H₃Cl₂F | dichloro-fluoro-propene |
| HCFO-1242zf | CH₂═CH—CClF₂ | 3-chloro-3,3-difluoropropene |
| HCFC-353jff | CCl₃CH₂CH₂CF₃ | 1,1,1-trichloro-4,4,4-trifluorobutane |
| HCFC-343jmz | CCl₃CH(CH₂Cl)CF₃ | 1,1,1-trichloro-2-(chloromethyl)- |
| | | 3,3,3-trifluoropropane |
| HCFC-252da | CH₂ClCHClCHF₂ | 1,2-dichloro-3,3-difluoropropane |
| HCFC-253fb | CH₂ClCH₂CF₃ | 1-chloro-3,3,3-trifluoropropane |
| HCC | | hydrochlorocarbon |
| CFC | | chlorofluorocarbon |
| HCFC | | hydrochlorofluorocarbon |
| HFC | | hydrofluorocarbons |
| HCFO | | hydrochlorofluoroolefin |
| HFO | | hydrofluoroolefin |
| GWP | | global warming potential |
| POE | | polyol ester |
| PAG | | polyalkylene glycol |
| PVE | | polyvinyl ether |
| PFPE | | perfluoropolyether |

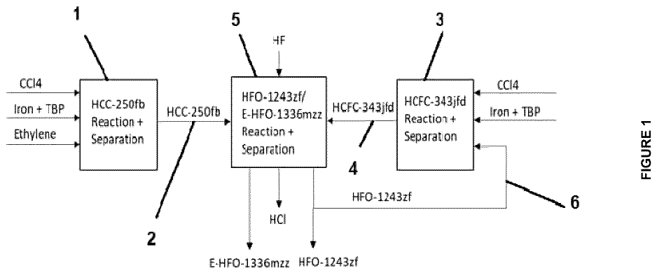

As shown in FIG. 1, HCC-250fb and HCFC-343jfd streams fed to reactor 5 are separately produced, each involving the reaction of a hydro(halo)carbon, e.g., CCl₄and an alkene or fluoroalkene, e.g., ethylene and 1,1,1-trifluoropropene, produced in separate (discrete) reactors 1 and 3 where HCC-250fb and 343jfd are respectively formed. HCC-250fb and HCFC-343jfd product streams are respectively separated into feed streams 2 and 4 using conventional techniques and introduced into common reactor 5. Reactor 5 contains, for example, a Cr₂O₃ catalyst (not shown) where the HCC-250fb, HCFC-343jfd and hydrogen fluoride (HF) contact the catalyst and hydrofluorinate and convert HCC-250fb and HCFC-343jfd into HFO-1243zf and HFO-E-1336mzz. Suitable Cr₂O₃ catalysts include, but are not limited to, JM 62-2 (chrome catalyst available from Johnson Matthey), LV(chrome catalyst available from The Chemours Company), JM-62-3 (chrome catalyst available from Johnson Matthey), and Newport Chrome (chrome catalyst available from Chemours). The chromium catalysts are preferably activated before use, typically by a procedure whereby the catalyst is heated to from 350° C. to 400° C. under a flow of nitrogen for a period of time, after which the catalyst is heated under a flow of HF and nitrogen or air for an additional period of time. HFO-E-1336mzz is separated and withdrawn for purification. HFO-1243zf is also separated and recycled back to reactor 3 via recycle stream 6.

### Stream 2

Although 250fb (CCl₃CH₂CH₂Cl, GC purity 100%) is commercially available from Chemours LLC, in some embodiments the CCl₃CH₂CH₂Cl can be prepared by the addition reaction of ethylene and carbon tetrachloride (CCIa) to produce 1,1,1,3-tetrachloropropane (HCC-250fb) in one of the vapor phase or the liquid phase according to the processes described on U.S Patent and Publication U.S. Pat. Nos. 4,605,802; 5,705,779; ¶[0023]-¶[0027] of 20110237843A1; and ¶[0018] through ¶[0022], ¶[0031] through ¶[0033], ¶[0037], ¶[0040], ¶[0045] through ¶[0056] and ¶[0114] of 2019/0233353A1, the entire disclosure of each incorporated herein by reference in its entirety.

In some embodiments the CCl₃CH₂CH₂Cl can be prepared in reactor 1 by conducting the reaction according to the process described in ¶[0018] through ¶[0022], ¶[0031] through ¶[0033], ¶[0037], ¶[0040], ¶[0045] through ¶[0056] and ¶[0114] of U.S. Patent Publication No. 2019/0233353A1, the disclosure of which is incorporated herein by reference in its entirety.

In some embodiments, the CCl₃CH₂CH₂Cl (250fb) is prepared by contacting carbon tetrachloride (CCl₄), ethylene (CH₂═CH₂), and a metal-ligand catalyst formed from metals, e.g., selected from the group consisting of Fe, Co, Ni, Cu, Mo, Cr, and Mn. In one embodiment iron metal, including but not limited to iron powder, iron wire, iron screen or iron turnings which form an organophosphine (organophosphorus) compound.

In certain embodiments, the CCl₃CH₂CH₂Cl (250fb) can be prepared according to the process described in U.S. Pat. No. 4,605,802 wherein the reaction is effected by adding carbon tetrachloride, phosphite and iron powder at about 70° C. to 140° C. and from about 25 to 500 psig (0.17 to 3.45 MPa), and the iron powder is employed in excess of about 0.001 mole per mole of carbon tetrachloride, the disclosure of which is incorporated herein by reference in its entirety.

In certain embodiments, the CCl₃CH₂CH₂Cl (250fb) could also be prepared according to the process described in U.S. Pat. No. 5,705,779 incorporated herein by reference.

In certain embodiments, the CCl₃CH₂CH₂Cl (250fb) can be prepared according to the process described in Example 6 of U.S. Pat. No. 4,605,802 which is incorporated herein by reference. In some embodiments, the organophosphine ligand may be an alkylphosphine or arylphosphine, including but not limited to triphenyl phosphine, tributyl phosphine and the like. In one embodiment, the phosphine ligand comprises triphenylphosphine such as triphenylphosphate. In another embodiment, the phosphine ligand consists essentially of triphenylphosphine such as triphenylphosphate. In another embodiment, the phosphine ligand consists of triphenylphosphine such as triphenylphosphate.

In one embodiment, the CCl₄/ethylene reaction may be carried out at an elevated temperature. In another embodiment, the CCl₄/ethylene reaction may be carried out at a temperature between about 50° C. and 250° C. In another embodiment, the CCl₄/ethylene reaction may be carried out at a temperature between about 100° C. and 200° C. In another embodiment, the CCl₄/ethylene reaction may be carried out at a temperature between about 120° C. and 180° C. In another embodiment, the CCl₄/ethylene reaction may be carried out at a temperature between about 130° C. and 170° C.

In some embodiments of the invention, the reaction in reactor 1 involves a metal catalyzed olefin insertion process that includes using a metal, e.g., selected from Fe, Co, Ni, Cu, Mo, Cr, and Mn and a ligand to obtain a desired product, such as a haloalkane insertion product, by insertion of an olefin into a haloalkane reactant. In particular, some aspects are directed to an iron and trialkyl phosphine catalytic system for olefin insertion of haloalkanes with a high rate of conversion and selectivity.

In certain embodiments, the metallic iron component of the catalyst may be from any source (including a combination of sources) of an iron component and may be iron powder, iron wire, iron sieve, or iron filings.

In some embodiments, the organophosphorus ligand may be an alkylphosphate or arylphosphate including but not limited to triphenyl phosphate, tributyl phosphate and the like. In one embodiment, the phosphate ligand comprises triphenylphosphate. In another embodiment, the phosphate ligand consists essentially of triphenylphosphate. In another embodiment, the phosphate ligand consists of triphenylphosphate (TPP) as a liquid phase process in the presence of metallic iron and a phosphine comprising tributyl phosphate (TBP).

In one embodiment, each component of the iron and phosphine catalytic systems has a particular concentration with respect to the moles of olefinic reactant used. As such, in some embodiments, a ratio of the number of moles of halocarbon reactant to moles of olefin is from about 3:1 to 1:1. In another embodiment, the molar ratio of halocarbon reactant to moles of olefin is from about 2.25:1 to 1:1. In another embodiment, the molar ratio of halocarbon reactant to moles of olefin is from about 2:1 to 1.1.

In one embodiment, a ratio of the number of moles of iron to the number of moles of olefin is from about 0.01:1 to 0.1:1. In another embodiment, the molar ratio of iron to olefin is from about 0.03:1 to 0.06:1. In another embodiment, the ratio of iron to olefin is from about 0.07:1 to 0.1:1.

In another embodiment, the number of moles of phosphine ligand may be measured in relation to a number of moles of olefin present in the reaction system. For example, in one embodiment, a molar ratio of phosphine ligand to olefin may be from about 0.01:1 to 0.04:1. In another embodiment, the molar ratio of phosphine ligand to olefin may be from about 0.02:1 to 0.06:1. For example, the molar ratio of phosphine ligand to olefin may be 0.023:1, while in another example, the molar ratio of phosphine ligand to olefin may be 0.046:1.

In one embodiment, the reaction of CCl₄ and CH₂═CH₂, in the presence of iron and an alkyl or aryl phosphine may be carried out at an elevated temperature. In another embodiment, the reaction may be carried out at a temperature between about 50° C. and 250° C. In another embodiment, the reaction may be carried out at a temperature between about 75° C. and 150° C. In another embodiment, the reaction may be carried out at a temperature between about 100° C. and 200° C.

In certain embodiments, the reaction of CCl₄and CH₂═CH₂, in the presence of iron and an alkyl or aryl phosphine, may be carried out at a temperature between one of 50° C., 60° C., 70° C., 80° C., 90° C. and one of 110° C., 120° C., 130° C., 140° C., 150° C., 160° C.

In certain embodiments, the reaction of CCl₄and CH₂═CH₂, in the presence of iron and an alkyl or aryl phosphate may be carried out in reactor 1 at a pressure in the reaction zone of the reactor of from about 0 psig to 200 psig (0 MPa to 1.4 MPa). In another embodiment, the pressure in the reaction zone can be from about 30 psig to 180 psig (0.21 to 1.2 MPa). In certain embodiments the pressure in the reaction zone of reactor 1 may be carried out at a pressure between one of 10 psig, 20 psig, 30 psig, 40 psig, 50 psig, 60 psig, 70 psig, 80 psig and one of 100 psig, 110 psig, 120 psig, 130 psig, 140 psig, 150 psig, 160 psig, 170 psig, 180 psig, 190 psig and 200 psig (0.1 MPa, 0.14 MPa, 0.21 MPa, 0.28 MPa, 0.34 MPa, 0.41 MPa, 0.48 MPa, 0.55 MPa and one of 0.69 MPa, 0.76 MPa, 0.83 MPa, 0.90 MPa, 0.97 MPa, 1.0 MPa, 1.1 MPa, 1.2 MPa, 1.2 MPa 1.3 MPa and 1.4 MPa). In other embodiments the pressure in reactor 1 may be between greater than 0 and less than 1 atm, 1 atm, 2 atm, 3 atm, 4 atm, 5 atm, 6 atm, 7 atm, 8 atm up to and including about 15 atm.

In certain embodiments, the reaction of CCl₄ and CH₂═CH₂, in the presence of iron and an alkyl or aryl phosphate, may be carried out at a temperature between one of 50° C., 60° C., 70° C., 80° C., 90° C. and one of 110° C., 120° C., 130° C., 140° C., 150° C., 160° C. and at a pressure between one of 10 psig, 20 psig, 30 psig, 40 psig, 50 psig, 60 psig, 70 psig, 80 psig and one of 100 psig, 110 psig, 120 psig, 130 psig, 140 psig, 150 psig, 160 psig, 170 psig, 180 psig, 190 psig and 200 psig (0.1 MPa, 0.14 MPa, 0.21 MPa, 0.28 MPa, 0.34 MPa, 0.41 MPa, 0.48 MPa, 0.55 MPa and one of 0.69 MPa, 0.76 MPa, 0.83 MPa, 0.90 MPa, 0.97 MPa, 1.0 MPa, 1.1 MPa, 1.2 MPa, 1.2 MPa 1.3 MPa and 1.4 MPa).

### Stream 4

In some embodiments, CF₃CHClCH₂CCl₃ (HCFC-343jfd) is prepared in reactor 3 according to the process described in ¶[0032] through ¶[0042] of U.S. Patent Publication No. 20190077733, the disclosure of which is incorporated herein by reference in its entirely and describe processes comprising contacting carbon tetrachloride with 1,1,1-trufluoropropene (HFO-1243zf) in the presence of an organophosphorus compound and a catalyst comprising a metal.

In one embodiment, CF₃CHClCH₂CCl₃ (HCFC-343jfd) is prepared in reactor 3 according to the process described ¶[0017] through ¶[0020] and ¶ [0032] through-¶[0044] of U.S. Patent Publication No. 20190077733, the disclosure of which is incorporated herein by reference in its entirely. In several embodiments carbon tetrachloride and 3,3,3-trifluoropropene contact a metal catalyst, and a phosphorus compound wherein the metal comprises one of is iron (Fe), cobalt (Co), nickel (Ni), copper (Cu), molybdenum (Mo) chromium (Cr), and manganese (Mn) and the phosphorus compound comprises one of a phosphate ester, a phosphate amide, a phosphonic acid, a phosphonic ester, a phosphinic acid, a phosphinic ester, a phosphine oxide, a phosphine imide, a phosphonium salt, a phosphorene, a phosphite, a phosphonate, a phosphinite, and a phosphine.

In some embodiments, the organophosphorus compound is selected from the group comprising, consisting essential of or consisting of a phosphate, a diphosphate, a triphosphate, and a trialkylphosphate. In some embodiments, the organophosphorus compound is tributylphosphate.

In some embodiments, CF₃CHClCH₂CCl₃ (343jfd) is produced by contacting carbon tetrachloride with 3,3,3-trifluoropropene in the presence of an organophosphorus compound and a catalyst selected from Fe, Co, Ni, Cu, Mo, Cr, and Mn. In some embodiments, the metal is Fe.

In some embodiments, CF₃CHClCH₂CCl₃ (343jfd) is produced by contacting carbon tetrachloride with 3,3,3-trifluoropropene at a temperature of from about 100° C. to about 120° C.

In some embodiments, reacting carbon tetrachloride in a liquid phase with 3,3,3-trifluoropropene in the presence of an organophosphorus compound and a catalyst comprising a metal to produce CF₃CHClCH₂CCl₃ (343jfd) is performed at a temperature of about 90° C. to about 130° C., about 100° C. to about 120° C., or about 105° C. to about 115° C. In some embodiments, reacting carbon tetrachloride in a liquid phase with 3,3,3-trifluoropropene in the presence of an organophosphorus compound and a catalyst comprising a metal to produce CF₃CHClCH₂CCl₃ (343jfd) is performed at a temperature of about 90° C., 95° C., 100° C., 105° C., 110° C., 115° C., 120° C., 125° C., or about 130° C.

In some embodiments, the organophosphorus compound is selected from the group consisting of a phosphate ester, a phosphate amide, a phosphonic acid, a phosphonic ester, a phosphinic acid, a phosphinic ester, a phosphine oxide, a phosphine imide, a phosphonium salt, a phosphorene, a phosphite, a phosphonate, a phosphinite, and a phosphine. In some embodiments, the organophosphorus compound is selected from a phosphate, a diphosphate, a triphosphate, and a trialkylphosphate.

In some embodiments, the organophosphorus compound is a phosphate, a diphosphate, a triphosphate, and a trialkylphosphate. In some embodiments, the organophosphorus compound is tributylphosphate.

In some embodiments, the metal of the catalyst for producing 343jfd is selected from the group consisting of Fe, Co, Ni, Cu, Mo, Cr, and Mn. In some embodiments, the metal is Fe.

In some embodiments, producing 343jfd is conducted at a temperature of from about 100° C. to about 120° C.

In some embodiments, the mole ratio of CCl₄ and 1,1,1-trifluoropropene (HFO-1243zf) is greater than 1:1, including but not limited to 1.5:1, 2:1, 2.5:1, 3:1 and all values and ranges therebetween.

In some embodiments, at least some of the HFO-1243zf is sourced from, and more particularly separated and recycled from the common reactor 5 via a recycle stream 6 to reactor 3 where it is contacted with CCl₄ to form HCFC-343jfd.

### Reactor 5

In some embodiments, the 250fb:343jfd feed mole ratio to reactor 5 is selected from one of 9:11 to 11:9, 0.45 to 0.55, and between 0.55 to 0.45.

In certain embodiments, the mole ratio of 250fb:343jfd is about 1:1.

In certain embodiments, the conversion of CCl₃CH₂CH₂Cl and/or 1,1,3-trichloro-4,4,4-trifluorobut-1-ene in reactor 5 is one of at least 95%, at least 96%, at least 98%, at least 99% and 100%.

In certain embodiments the product mixture of reactor 5 comprises HFO-1234z, HFO-E-1336mzz, and optionally one or more of 346mdf, 253fb, 1242zf, 1336 ft, 356mff, Z-1326mxz, 1233xf, E-1233zd, 252da, 1241 isomer, and 1333azd.

In certain embodiments, the HFO-1234zf in product mixture of reactor 5 comprises about 45 mole percent, about 50 mole percent, about 55 mole percent, between 40 mole percent and 60 mole percent, between 40 mole percent and 55 mole percent, between 45 mole percent and 55 mole percent.

In certain embodiments, the HFO-E-1336mzz in product mixture of reactor 5 comprises about 45 mole percent, about 50 mole percent, about 55 mole percent, between 40 mole percent and 60 mole percent, between 40 mole percent and 55 mole percent, between 45 mole percent and 55 mole percent.

In certain embodiments, the HFO-Z-1336mzz in the product mixture comprises less than 1 mole percent, 0.9 mole percent 0.8 mole percent, 0.7 mole percent, 0.6 mole percent, 0.5 mole percent 0.4 mole percent, 0.3 mole percent 0.2 mole percent, 0.1 mole percent, greater than 0.3, 0.4, 0.5, 0.6 or 0.7 but less than 1 mole percent, between greater than zero and less than 1 mole percent, between 0.1, 0.2, 0.3, 04, 0.5, 0.6, 0.7, 0.8, or 0.9 and 1.0 mole percent, between 0.2, 0.3, 04, 0.5, 0.6, 0.7, or 0.8, or and 0.9 mole percent, between 0.2, 0.3, 04, 0.5, 0.6, or 0.7 and 0.8 or 0.9 mole percent, between 0.3, 04, 0.5, 0.6, or 0.7 and 0.8 or 0.9 mole percent, between 0.3, 04, 0.5, or 0.6 and 0.7, 0.8 or 0.9 mole percent, and all ranges and values between >0 and <1 mole percent.

In certain embodiments, E-HFO-1336mzz and HFO-1243zf are respectively isolated from the product mixture.

In certain embodiments disclosed herein, the E-HFO-1336mzz from the product mixture is processed for use as at least part of a working fluid in heat transfer systems including, but are not limited to, air conditioners (e.g., automotive air conditioners), freezers, refrigeration machines, heat pumps, chillers (e.g., water chillers, flooded evaporator chillers, direct expansion chillers, centrifugal chillers), walk-in chillers, high temperature heat pumps, mobile chillers, mobile air conditioning units, immersion cooling systems, data center cooling systems, and combinations thereof, and as a component of compositions for heat transfer, refrigeration, high-temperature heat pumps, immersion cooling systems, organic Rankine cycles, fire extinguishing/fire suppression agents, propellants, foam blowing agents, solvents, and/or cleaning fluids.

In certain embodiments disclosed herein, E-HFO-1336mzz, alone or in blends, is used as a working fluid in heat transfer systems including, but are not limited to, air conditioners (e.g., automotive air conditioners), freezers, refrigeration machines, heat pumps, chillers (e.g., water chillers, flooded evaporator chillers, direct expansion chillers, centrifugal chillers), walk-in chillers, high temperature heat pumps, mobile chillers, mobile air conditioning units, immersion cooling systems, data center cooling systems, and combinations thereof, and as a component of compositions for heat transfer, refrigeration, high-temperature heat pumps, immersion cooling systems, organic Rankine cycles, fire extinguishing/fire suppression agents, propellants, foam blowing agents, solvents, and/or cleaning fluids.

In certain embodiment disclosed herein, the E-HFO-1336mzz of the product mixture is used as working fluid in a heat exchanger and, thereby, producing a cooled working fluid.

### EXAMPLES

The present disclosure is further defined in the following Examples. It should be understood that these Examples, while indicating preferred embodiments, are given by way of illustration only. From the above discussion and these Examples, one skilled in the art can ascertain the preferred features, and without departing from the spirit and scope thereof, can make various changes and modifications to adapt it to various uses and conditions.

### Example 1: Compound 343Jfd Preparation

3,3,3-trifluoropropene (66 g, 0.68 mol) was added to a mixture of carbon tetrachloride (158 g, 1.0 mol), Fe powder (1.12 g, 0.02 mol) and tributylphosphate (2.66, 0.01 mol) in a 400 mL Hastelloy reactor. The reactor was heated up to 110° C. for 3 hours. 217 g mixture was transferred to a container and analyzed by GC (100% TFP conversion, 88% selectivity to 343jfd). The same reaction was repeated twice and all three batches of the material were combined. The subsequent fractionation provided 299 g 98.5% pure CCl₃CH₂CHClCF₃ (343jfd). CCl₃CH₂CHClCF₃: b.p. 92~94 oC/140 torr; ¹H NMR (CDCl3, 400 MHz) δ 4.52 (1 H, q-d-d, J1=J2=6.9 Hz, J3=1.8 Hz), 3.44 (1H, d-d, J1=16.0 Hz, J2=1.9 Hz), 3.26 (1H, d-d, J1=16.0 Hz, J2=7.6 Hz); ¹⁹F NMR (CDCl3, 376 MHz) δ −74.85 (3F, d, J=6.9 Hz); MS (El): 213 (M⁺-Cl).

### Example 2: Example for Coproducing 1234zf and E-1336Mzz

An Inconel® pipe (0.5 inch (1.27 cm) OD, 10 inch (25.4 cm) length, 0.35 in (0.89 cm) (wall thickness) was the reactor and was filled with 6 cc of JM 62-3 Cr₂O₃ catalyst obtained from Johnson Matthey PLC. The reactor was heated to an elevated temperature, and the reaction(s) was carried out in the vapor phase.

A 1:1 mol mixture of 250fb and 343jfd was fed to the reactor at 0.15 mL/hr or 0.3 mL/hr via an ISCO pump via a vaporizer controlled at 180° C. along with HF. The pressure of the reaction was between 0 to 50 psig (0 to 0.345 MPa). The effluent of the reactor was analyzed online using an Agilent® 7890 GC/5971 MS. The conditions are summarized in Table 1 below. (Note: CT is contact time in seconds.) The results are provided in Table 2 below. Conversion of both 250fb and 343jfd were 100% in all samples.

TABLE 1
Coproduction of 1243zf and E-1336mzz
over a Chrome Catalyst - Conditions

| | Furnace | | | | | |
|---|---|---|---|---|---|---|
| | Temp | Pressure | Pressure | Pump | HF | CT |
| Sample | (° C.) | (psig) | (kPa) | (mL/hr) | (sccm) | (sec) |
| 1 | 300 | 0.15 | 1.03 | 0.15 | 8.14 | 18 |
| 2 | 300 | 0.15 | 1.03 | 0.15 | 8.14 | 18 |
| 3 | 300 | 0.15 | 1.03 | 0.15 | 8.14 | 18 |
| 4 | 300 | 0.12 | 0.82 | 0.15 | 8.14 | 18 |
| 5 | 300 | 0.12 | 0.82 | 0.15 | 8.14 | 18 |
| 6 | 325 | 0.10 | 0.69 | 0.15 | 7.87 | 18 |
| 7 | 325 | 0.09 | 0.62 | 0.15 | 7.87 | 18 |
| 8 | 325 | 0.09 | 0.62 | 0.15 | 7.87 | 18 |
| 9 | 325 | 0.08 | 0.55 | 0.15 | 7.87 | 18 |
| 10 | 325 | 0.09 | 0.62 | 0.3 | 7.87 | 18 |
| 11 | 300 | 0.12 | 0.82 | 0.3 | 17.23 | 9 |
| 12 | 300 | 0.12 | 0.82 | 0.3 | 17.23 | 9 |
| 13 | 300 | 0.12 | 0.82 | 0.3 | 17.23 | 9 |
| 14 | 300 | 0.11 | 0.76 | 0.3 | 17.23 | 9 |
| 15 | 300 | 0.11 | 0.76 | 0.3 | 17.23 | 9 |
| 16 | 325 | 0.11 | 0.76 | 0.3 | 16.38 | 9 |
| 17 | 325 | 0.14 | 0.97 | 0.3 | 16.38 | 9 |
| 18 | 325 | 0.16 | 1.10 | 0.3 | 16.38 | 9 |
| 19 | 325 | 0.16 | 1.10 | 0.3 | 16.38 | 9 |
| 20 | 325 | 0.15 | 1.03 | 0.3 | 16.38 | 9 |

TABLE 2
Coproduction of 1243zf and E-1336mzz
over a Chrome Catalyst - Results

| | 1243zf | E-1336mzz | 347mef | Z-1336mzz | Unknown |
|---|---|---|---|---|---|
| Sample | Mole Percent (mole %) | | | | |
| 1 | 46.53 | 50.52 | 0.31 | 0.50 | 2.14 |
| 2 | 45.26 | 51.75 | 0.31 | 0.51 | 2.17 |
| 3 | 45.66 | 51.46 | 0.30 | 0.51 | 2.07 |
| 4 | 45.91 | 51.18 | 0.29 | 0.51 | 2.11 |
| 5 | 46.11 | 51.28 | 0.37 | 0.51 | 1.74 |
| 6 | 44.69 | 52.32 | 0.53 | 0.61 | 1.84 |
| 7 | 44.69 | 51.94 | 0.55 | 0.61 | 2.21 |
| 8 | 45.13 | 52.08 | 0.57 | 0.61 | 1.60 |
| 9 | 45.35 | 51.95 | 0.59 | 0.61 | 1.50 |
| 10 | 44.90 | 51.81 | 0.56 | 0.23 | 2.50 |
| 11 | 51.33 | 45.41 | 0.26 | 0.46 | 2.54 |
| 12 | 43.76 | 52.84 | 0.34 | 0.53 | 2.53 |
| 13 | 46.02 | 50.96 | 0.33 | 0.51 | 2.18 |
| 14 | 46.57 | 50.21 | 0.32 | 0.50 | 2.40 |
| 15 | 46.56 | 50.36 | 0.32 | 0.50 | 2.26 |
| 16 | 46.57 | 50.59 | 0.57 | 0.60 | 1.67 |
| 17 | 46.17 | 50.53 | 0.63 | 0.60 | 2.07 |
| 18 | 46.79 | 50.16 | 0.51 | 0.59 | 1.95 |
| 19 | 46.97 | 50.36 | 0.50 | 0.60 | 1.58 |
| 20 | 46.30 | 50.49 | 0.49 | 0.37 | 2.35 |

Unknown components comprise at least one the following components: 346mdf, 253fb, 1242zf, 1336 ft, 356mff, Z-1326mxz, 1233xf, E-1233zd, 252da, 1241 isomer, and 1333azd.

The data of Table 1 shows that 1243zf and E-1336mzz can be co-produced over a chrome catalyst and the product stream includes HFO-1243zf, E/Z-HFO-1336mzz, and CF₃CFHCH2CF₃, heptafluorobutane (HFC-347mef).

### OTHER EMBODIMENTS

A process embodiment 1 of contacting CCl₃CH₂CH₂Cl (HCC-250fb) and CCl₃CH₂CHClCF₃ (343jfd) with a catalyst in the presence of hydrogen fluoride (HF) in a reaction zone of a reactor; and producing a product mixture comprising at least 1,1,1-trifluoropropene (HFO-1243zf), E-1,1,1,4,4,4-Hexafluoro-2-butene (E-1336mzz) and hydrogen chloride.

A process embodiment 2 including process embodiment 1 and first producing the CCl₃CH₂CH₂Cl (HCC-250fb) component in a first reaction zone of a reactor; first producing the CCl₃CH₂CHClCF₃ (343jfd)component in a second reaction zone of the reactor; respectively isolating the CCl₃CH₂CH₂Cl (HCC-250fb) and CCl₃CH₂CHClCF₃ (343jfd); and using the CCl₃CH₂CH₂Cl (HCC-250fb) and CCl₃CH₂CHClCF₃ (343jfd) as the feed of embodiment 1.

A composition embodiment 1 containing CCl₃CH₂CH₂Cl (HCC-250fb) and CCl₃CH₂CHClCF₃ (343jfd).

The composition embodiment 1 wherein the 250fb:343jfdmole ratio is between 0.5:1 to 2:1.

A composition embodiment 2 which is a product mixture of 1,1,1-trifluoropropene (HFO-1243zf) and E-1,1,1,4,4,4-Hexafluoro-2-butene (E-1336mzz).

The composition embodiment 2 comprising at least one of 253fb, 1242zf, 1336 ft, 356mff, Z-1326mxz, 1233xf, E-1233zd, 252da, 1241 isomer, and 1333azd.

The composition embodiment 2 comprising at least two of 346mdf, 253fb, 1242zf, 1336 ft, 356mff, Z-1326mxz, 1233xf, E-1233zd, 252da, 1241 isomer, and 1333azd.

The composition embodiment 2 comprising more than three of 346mdf, 253fb, 1242zf, 1336 ft, 356mff, Z-1326mxz, 1233xf, E-1233zd, 252da, 1241 isomer, and 1333azd.

The composition embodiment 2 further comprising at least one heptafluorobutane isomer.

Process embodiment 3 involves using E-HFO-1336mzz made from process embodiment 1 as a working fluid in heat transfer systems including, but are not limited to, air conditioners (e.g., automotive air conditioners), freezers, refrigeration machines, heat pumps, chillers (e.g., water chillers, flooded evaporator chillers, direct expansion chillers, centrifugal chillers), walk-in chillers, high temperature heat pumps, mobile chillers, mobile air conditioning units, immersion cooling systems, data center cooling systems, and combinations thereof, and as a component of compositions for heat transfer, refrigeration, high-temperature heat pumps, immersion cooling systems, organic Rankine cycles, fire extinguishing/fire suppression agents, propellants, foam blowing agents, solvents, and/or cleaning fluids.

The process of embodiment where HCC-250fb and/or 343jfd are produced in the liquid phase.

The process of embodiment where the liquid phase reaction is conducted at a temperature of 200° C. to 400° C.

The process of embodiment where the HCC-250fb:343jfd mole ratio between 0.1:0.9 to 0.9:0.1.

The process of embodiment where the 1,1,1-trifluoropropene (HFO-1243zf): E-1,1,1,4,4,4-hexafluoro-2-butene (E-1336mzz) mole ratio of between 0.1:0.9 to 0.9:0.1.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art (s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

1. A composition comprising CCl₃CH₂CH₂Cl (HCC-250fb) and CCl₃CH₂CHClCF₃ (343jfd) at a 250fb:343jfd mole ratio between 0.05:0.95 to 0.95:0.05.

2. The composition of claim 1 wherein the 250fb:343jfd mole ratio is 0.3:0.7 to 0.7 to 0.3.

3-7. (canceled)

8. A process comprising,
(a) contacting CCl₃CH₂CH₂Cl (HCFC-250fb) and CCl₃CH₂CHClCF₃ (343jfd) with a catalyst in the presence of hydrogen fluoride (HF) in a reaction zone of a reactor; and
(b) producing a product mixture comprising at least 1,1,1-trifluoropropene (HFO-1243zf), E-1,1,1,4,4,4-Hexafluoro-2-butene (E-1336mzz) and hydrogen chloride.

9. The process of claim 8 wherein the reactor is a single reactor.

10. The process of claim 8 wherein the contacting occurs in the vapor phase.

11. The process of claim 8 wherein the contacting occurs at a temperature between 275° C. to 350° C.

12. The process of claim 8 where the contact time is between 1 to 120 seconds.

13. The process of claim 8 wherein the contacting occurs at a pressure between atmospheric pressure to 300 psi.

14. The process of claim 8 wherein the contacting occurs at a temperature between 275° C. to 350° C. and a pressure of 50 to 200 psi.

15. The process of claim 8 wherein the contacting occurs at a temperature between 275° C. to 350° ca pressure of 60 to 180 psi and a contact time of 3 to 30 seconds.

16. The process of claim 8, further comprising:
(i) producing the CCl₃CH₂CH₂Cl (HCFC-250fb) in the liquid phase;
(ii) producing the CCl₃CH₂CHClCF₃ (343jfd) in the liquid phase; and
(iii) isolating the CCl₃CH₂CH₂Cl (HCFC-250fb) and CCl₃CH₂CHClCF₃ (343jfd); and
(iv) providing the CCl₃CH₂CH₂Cl (HCFC-250fb) and CCl₃CH₂CHClCF₃ (343jfd) as the feed of (a).

17-21. (canceled)

22. The process of claim 16 wherein the contacting of (a) is conducted at a temperature of 200 to 400° C.

23. The process of claim 16 wherein the feed of (a) comprises a CCl₃CH₂CH₂Cl (HCFC-250fb):CCl₃CH₂CHClCF₃ (343jfd) mole ratio between 0.1:0.9 to 0.9:0.1.

24. The process of claim 16 wherein the product mixture of (b) comprises 1,1,1-trifluoropropene (HFO-1243zf) and E-1,1,1,4,4,4-Hexafluoro-2-butene (E-1336mzz) in a mole ratio of between 0.1:0.9 to 0.9:0.1.

25-30. (canceled)

31. The process of claim 16 wherein (i) and (ii) are conducted in the presence of a catalyst.

32. The process of claim 31 wherein the catalyst comprises a phosphine catalytic system.

33. (canceled)

34. The process of claim 31 wherein the catalyst comprises one of an iron powder, iron wire, iron sieve, iron filings of combination thereof.

35. The process of claim 31 wherein the catalyst includes one of an alkyl or aryl phosphine and a metal.

36. The process of claim 35 wherein the metal comprises one of an iron powder, iron wire, iron sieve, iron filings of combination thereof.

37. A process comprising isolating E-HFO-1336mzz from a product mixture resulting from the concurrent fluorination of HCC-250fb and HCFC-343jfd, and using the isolated E-HFO-1336mzz as at least part of a working fluid, in a heat exchanger and, thereby, producing a cooled working fluid.

38-43. (canceled)
